(19) United States
(12) Patent Application Publication
(10) Pub. No.: US 2024/0252241 A1
(43) Pub. Date: Aug. 1, 2024

(54) METHODS AND DEVICES FOR ENABLING ACTIVE MONITORING AND COMMUNICATIONS BETWEEN MEDICAL FIBER OPTIC CATHETERS AND MEDICAL LASER LIGHT SYSTEMS

(71) Applicant: KONINKLIJKE PHILIPS N.V., EINDHOVEN (NL)

(72) Inventors: Jeffrey SCHINKEL, COLORADO SPRINGS, CO (US); Kenneth Peter GRACE, WOODLAND PARK, CO (US); Michael ANDERSON, COLORADO SPRINGS, CO (US); James David CEZO, COLORADO SPRINGS, CO (US)

(21) Appl. No.: 18/560,754

(22) PCT Filed: May 16, 2022
(86) PCT No.: PCT/EP2022/063142

§ 371 Date: Nov. 14, 2023

Related U.S. Application Data
(60) Provisional application No. 63/189,746, filed on May 18, 2021.

(51) Int. Cl. A61B 18/24; A61B 18/00

(52) U.S. Cl. CPC A61B 18/24; A61B 2018/00178; A61B 2018/00184

(57) ABSTRACT

A source-side coupler (10) for injecting light from an associated light source (4) into an associated fiber optic catheter (2) includes: an optical output aperture (14) configured to inject light from the associated light source into the associated fiber optic catheter; a plurality of mechanical switches (16) configured to be engaged by pins (34) of a catheter-side coupler; and an electronic processor (20) programmed to receive information from the associated fiber optic catheter via the connection by: electrically receiving the information if the connection establishes an electrical connection between the electronic processor and the associated fiber optic catheter, or mechanically receiving the information based on which mechanical switches of the plurality of mechanical switches are engaged by pins of the catheter-side coupler if the connection does not establish an electrical connection between the electronic processor and the associated fiber optic catheter.

### FIELD

The following relates generally to the catheter arts, catheter coupling arts, catheter detection arts, and related arts.

### BACKGROUND

Fiber optic catheters are used in a range of therapies, such as for performing atherectomies and other types of vascular procedures. In an atherectomy performed using a fiber optic catheter, the catheter is inserted into a large blood vessel and the tip is moved to a target location, and then an optical fiber or fiber bundle of the catheter carries high intensity light from a light source such as an excimer light source to the target location to ablate or otherwise remove atherosclerosis from the blood vessel at the target location. Similar therapies are sometimes also performed in veins. The catheter is usually a single-use medical accessory that attaches to a light source providing the laser light via a detachable coupling. The parameters of the laser light provided by the light source should be matched to optical transmission characteristics of the attached fiber optic catheter. To automate the matching process, it is advantageous for the light source to automatically recognize the catheter type of the fiber optic catheter when it is attached to the light source.

To this end, in some existing fiber optic catheter systems, a source (e.g., an excimer) laser-side coupler includes a bank of N mechanical switches. A mating catheter-side coupler includes molded pins that depress zero, all, or some subset of the N mechanical switches when the two couplers are mated together. Different types of catheters will have different subsets of molded pins. This approach conveys an N bit value to the laser, so that 2^{N} different types of catheters can be distinguished. Based on the type of catheter thusly identified, the laser can automatically configure the fluence rate, repetition rate, cycle time, and/or other operating parameters appropriate for that type of catheter using a look-up table that stores the operating parameters for each catheter type.

The following discloses certain improvements to overcome these problems and others.

### SUMMARY

In some embodiments disclosed herein, a source-side coupler is provided for injecting light from an associated light source into an associated fiber optic catheter via a connection of the source-side coupler with a catheter-side coupler of the associated fiber optic catheter. The source-side coupler includes an optical output aperture configured to inject light from the associated light source into the associated fiber optic catheter via the connection. A plurality of mechanical switches is configured to be engaged by pins of the catheter-side coupler during the connection, and at least two electrical lines. An electronic processor is programmed to receive information from the associated fiber optic catheter via the connection by: electrically receiving the information if the connection establishes an electrical connection between the electronic processor and the associated fiber optic catheter via the at least two electrical lines, or mechanically receiving the information based on which mechanical switches of the plurality of mechanical switches are engaged by pins of the catheter-side coupler if the connection does not establish an electrical connection between the electronic processor and the associated fiber optic catheter via the at least two electrical lines.

In some embodiments disclosed herein, a fiber optic catheter apparatus includes a catheter including an optical fiber or optical fiber bundle terminating at an optical output aperture. A catheter-side coupler includes a plurality of electrically conductive pins. An electronic processor is programmed transmit information about the fiber optic catheter apparatus to an associated light source in response to an electrical connection of the catheter-side coupler with a source-side coupler of the associated light source via the electrically conductive pins. A physical configuration of the plurality of electrically conductive pins encodes at least a portion of the information about the fiber optic catheter apparatus.

In some embodiments disclosed herein, a light system for injecting light into a fiber optic catheter includes a light source. A fiber optic catheter apparatus includes a catheter including an optical fiber or optical fiber bundle terminating at an optical output aperture. A catheter-side coupler includes a plurality of electrically conductive pins. A fiber optic catheter electronic processor is disposed in the catheter-side coupler and programmed transmit information about the fiber optic catheter apparatus to the light source. A physical configuration of the plurality of electrically conductive pins encodes at least a portion of the information about the fiber optic catheter apparatus. A source-side coupler is configured to inject light from the light source into the fiber optic catheter apparatus via a connection of the source-side coupler with the catheter-side coupler. The source-side coupler includes an optical coupler configured to inject light from the light source into the fiber optic catheter via the connection. A plurality of mechanical switches is configured to be engaged by the electrically-conductive pins of the catheter-side coupler during the connection, and at least two electrical lines. A source-side electronic processor is programmed to receive information from the fiber optic catheter via the connection by electrically receiving the information if the connection establishes an electrical connection between the source-side electronic processor and the fiber optic catheter via the at least two electrical lines, or mechanically receiving the information based on which mechanical switches of the plurality of mechanical switches are engaged by pins of the catheter-side coupler if the connection does not establish an electrical connection between the electronic processor and the fiber optic catheter via the at least two electrical lines.

One advantage resides in determining a type of catheter based on a configuration of an engagement between a source side coupler and a catheter coupler.

Another advantage resides in determining a type of catheter based on a configuration of an engagement between a source side coupler and a catheter coupler based on an electrical connection between the source side coupler and the catheter coupler.

Another advantage resides in determining a type of catheter based on an electrical connection as noted above, but alternatively based on a mechanical connection between the source side coupler and the catheter coupler if the catheter or light source are not designed to employ the electrical connection.

Another advantage resides in determining operating parameters of catheter based on a configuration of an engagement between a source side coupler and a catheter coupler.

Another advantage resides in determining when a catheter has been used and issuing a notification regarding a used catheter.

Another advantage resides in measuring an output energy of a laser from a light source through a catheter.

Another advantage resides in digitizing analog catheter measurements to reduce noise in the measurements.

Another advantage resides in measuring a temperature measurement of an ambient environment and compensating for the temperature during an intravascular procedure.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a catheter light system in accordance with the present disclosure.

FIG. 2 diagrammatically illustrates a coupler of the system of the system of FIG. 1.

FIG. 3 diagrammatically illustrates another coupler of the system of the system of FIG. 1.

FIG. 4 diagrammatically illustrates the coupler of FIG. 2 and the coupler of FIG. 3 engaged with each other.

FIG. 5 diagrammatically illustrates a catheter method using the system of FIG. 1-4.

### DETAILED DESCRIPTION

It is recognized herein that there can be some deficiencies in the approach of employing mechanical pins and mating switches to identify the catheter type. First, the number of different types of lasers that can be distinguished is 2^{N}, and for a reasonable number of mechanical switches (e.g., N=6) this is a relatively small number (e.g., 2⁶=64 types). Second, updating the operating parameters for a particular type of catheter entails updating a lookup table storing the operating parameters for each catheter type at the laser. Also, if two catheters of the same type (as indicated by the molded pins) have different operating parameters, there is no way to accommodate this.

On the other hand, the deployed base of excimer lasers that use the existing mechanical switches/molded pins approach is large. While a fiber optic catheter is typically a single-use device due to sanitation considerations, light sources of the excimer lasers can be used multiple times. Hence, it is recognized herein that it would be advantageous for any improved catheter to be backward compatible with existing (primarily excimer) light sources that are designed to identify catheter type based on mated molded pins.

The following discloses an approach for providing a “smart” catheter which is also backward compatible with the existing mechanical switches-based laser-side coupler. The disclosed approach adds electronics to the catheter-side coupler and replaces the molded plastic pins with electrically conductive metal pins (either by wholesale replacement or by providing molded plastic pins with electrically conductive coatings). The laser-side coupler still has the mechanical switches but also includes electrical wires attached to those switches. Hence, if a “dumb” catheter is plugged into a “smart” excimer laser (or vice versa) then the catheter-type identification works as usual, relying on the mechanical switches being depressed to convey information on the type of catheter being plugged into the laser.

However, if both the catheter and the laser are “smart”, then the smart laser detects the presence of a smart catheter based on electrical signals conveyed by the catheter electronics via the metal pins. Once identified as a smart catheter, information stored in memory of the catheter electronics can be transmitted to the smart laser. This can be done in wired fashion using the metal pins, or can be done wirelessly, e.g., by reading an RFID tag of the smart catheter. Information can also optionally be conveyed from the laser to the catheter.

The following discloses numerous use cases for the smart catheter design. In one approach, the memory of the catheter electronics stores the operating parameter values for the catheter, and these are conveyed to the laser. This simplifies upgrading a particular catheter type, as it requires only storing the correct (updated) operating parameters in the memory of the catheter prior to shipment.

In some embodiments disclosed herein, a photodiode can be added to the catheter-side coupler that measures the laser power transmitted through the catheter-side coupler. This can be done by arranging the photodiode to measure light that is leaked from the side of the optical fiber and applying a suitable calibration to extract the actual laser power. Optionally, a temperature sensor can also be included in the catheter-side coupler to refine laser power measurement accuracy if the photodiode output is overly temperature-dependent. Furthermore, to reduce noise the catheter electronics can include an analog-to-digital (A/D) converter to digitize the power reading before sending it to the laser.

In some embodiments disclosed herein, since the catheter is a single-use disposable item, the catheter electronics can store in memory a “used” flag that is set once the catheter is used. This flag value is sent to the laser, and if the “used” flag is set then the laser will not operate. Similarly, if the laser detects a fault condition during a procedure, it can write diagnostic information to the memory of the catheter electronics, which can be used to diagnose the problem when the faulty catheter is sent back to the manufacturer for analysis.

In some embodiments disclosed herein, a validation code can be conveyed from the catheter electronics to the laser, that is used to validate the catheter for use with the laser.

As previously noted, there are relatively few pins on the catheter-side coupler (e.g., a maximum of N pins corresponding to the N mechanical switches of the laser-side coupler, where N=6 in some systems). This is somewhat limiting since two pins will be used to convey electrical power to the catheter electronics. Hence, the wired communication preferably uses a serial method such as Inter-Integrated Circuit (I2C) serial communication. Alternatively, as previously noted, wireless communication could be used.

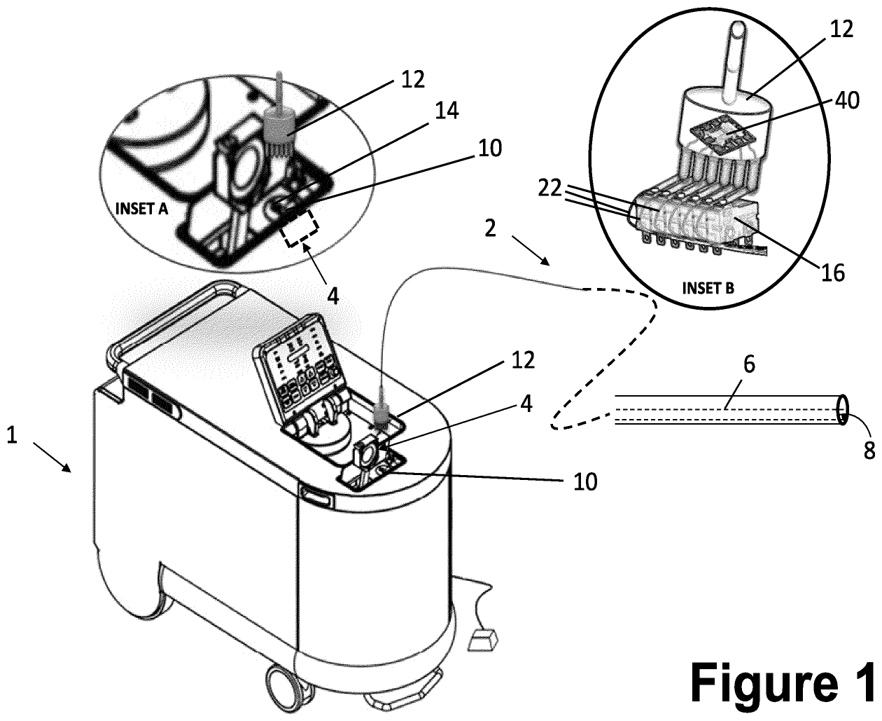

With reference to FIG. 1, an illustrative light system 1 for use with a catheter 2 is shown. As shown in FIG. 1, the light system includes one or more light sources 4 configured to supply or inject light into a fiber optic catheter 2. The catheter 2 is diagrammatically shown with a dashed line indicating most of the length of the catheter, and the end of the catheter shown in enlarged diagrammatic fashion to illustrate that the catheter 2 includes an optical fiber 6 (which may be a fiber bundle) terminating at an optical output aperture 8. The light system 1 also include a source-side (i.e., excimer) coupler 10 configured to engage or connect with a catheter-side coupler 12 of the catheter 2, as best shown in INSET A of FIG. 1. During an atherectomy or other vascular procedure, laser light from the light system 1 passes through the coupler 10 into a mating coupler 12 of the catheter 2 and is carried by the optical fiber or fiber bundle 6 and emitted at the optical output aperture 8 to ablate or otherwise remove atherosclerosis from a blood vessel at a target location.

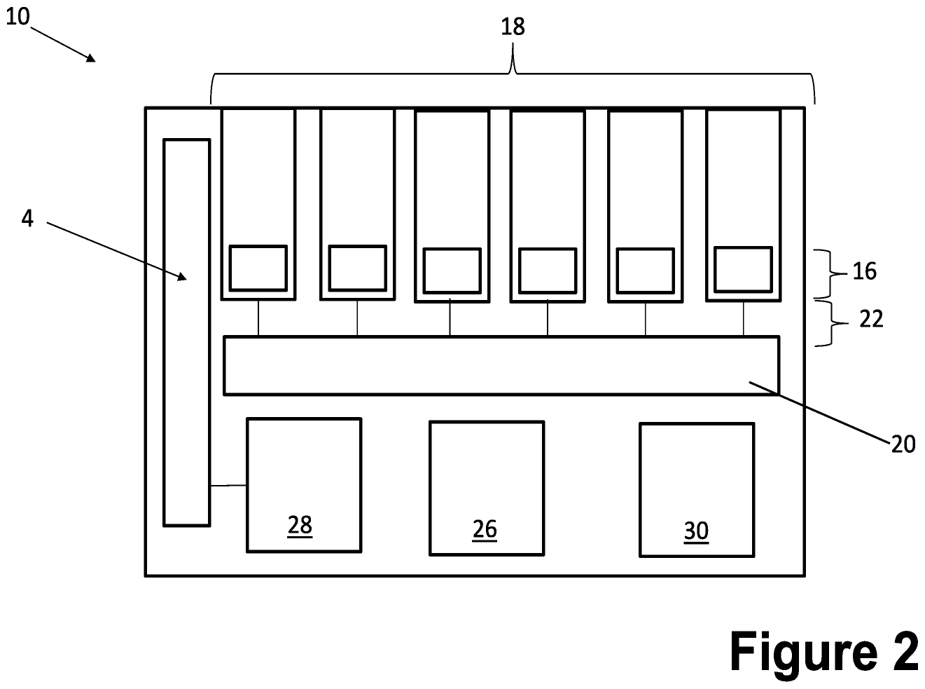

FIG. 2 shows an example of the source-side coupler 10. The source-side coupler 10 includes an optical output aperture 14 configured to inject light from the light source 4 into the fiber optic catheter 2 via the connection between the source-side coupler 10 and the catheter-side coupler 12. The source-side coupler 10 also includes a plurality of mechanical switches 16, in which each switch 16 is encased within a corresponding conductive socket 18 configured to be engaged by pins (not shown in FIG. 2) of the catheter-side coupler 12 during the connection. Alternatively, as shown in Inset B, the switches 16 can be constructed as a bank of switches. A source-side electronic processor 20 is included in the source-side coupler 10 and is programmed to receive information from the fiber optic catheter 2 via the connection. To do so, in one example, two or more electrical lines 22 are included, and if the connection establishes an electrical connection between the source-side electronic processor 20 and the fiber optic catheter 2 via the at least two electrical lines 22. Inset B of FIG. 1 illustrates one non-limiting illustrative approach for providing the electrical lines 22. Inset B also diagrammatically illustrates a catheter-side electronic processor 40 that suitably conveys the information to the source-side electronic processor 20. However, this assumes that both the light source 1 and the catheter 2 are equipped (e.g., with the processors 20, 40 and associated electrical connectors) to transfer the information via the electrical connection. If, however, this is not the case, then the information is mechanically received by the source-side electronic processor 20 based on which mechanical switches of the plurality of mechanical switches 16 are engaged by pins of the catheter-side coupler 12 if the connection does not establish an electrical connection between the source-side electronic processor 20 and the fiber optic catheter 2 via the at least two electrical lines 22. In a further example, the source-side coupler 10 includes a wireless radio receiver or transceiver 26, and the source-side electronic processor 20 is programmed to electrically receive the information via the wireless radio receiver or transceiver 26.

The source-side coupler 10 also includes a power supply 28 for power to be supplied thereto. In some examples, the power supply 28 can include a DC-to-DC converter which reduces the power supplied to the source-side coupler 10 from 12 Volts to 3.3 Volts. This is done in order to ensure that the source-side coupler 10 has plenty of power, even if the conductivity of the conductive sockets 18 degrades overtime. In addition, the source-side coupler 10 also includes a serial communication circuit 30, such as an I2C communication circuit, configured to communicate with a corresponding serial communication circuit (not shown in FIG. 2) of the catheter-side coupler 12. In some variant embodiments, the source-side coupler 10 can include printed traces (not shown) that do not connect with any pins of the catheter-side coupler 12, but do make electrical contact with traces (not shown) elsewhere in the catheter-side coupler 12. For example, electrical traces could be disposed on sidewalls of a socket and plug of a socket-and-plug connection arrangement. These electrical traces suitably replace the wires 22 shown in Inset B.

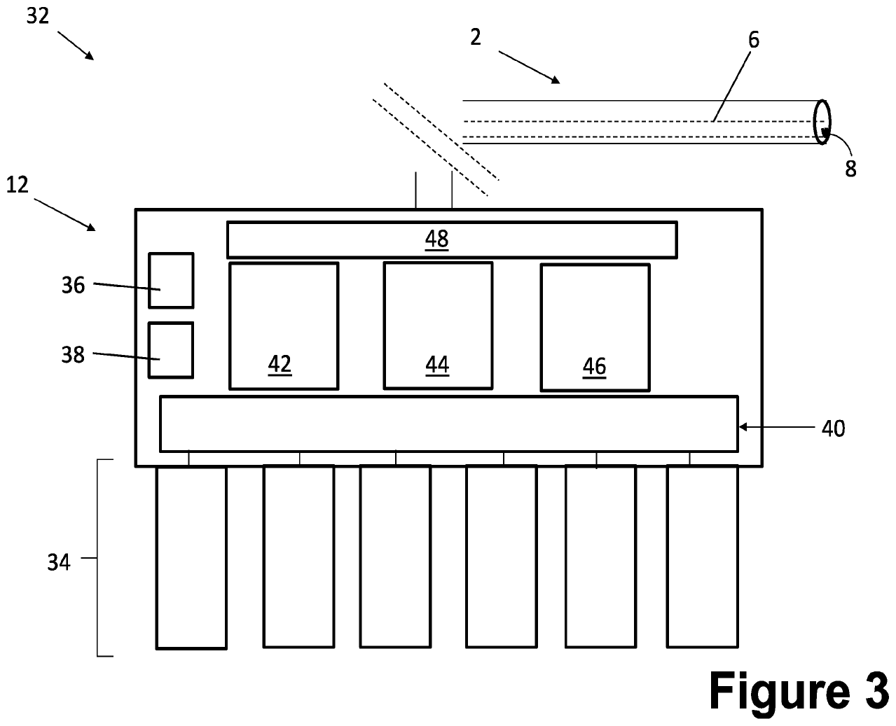

FIG. 3 shows an example of a fiber optic catheter apparatus 32 that includes the catheter-side coupler 12 and the catheter 2. As again shown in FIG. 3, the catheter 2 includes the optical fiber or fiber bundle 6 terminating at the optical output aperture 8. The catheter-side coupler 12 includes a plurality of electrically conductive pins 34 configured to engage corresponding mechanical switches 16 on the source-side coupler 10. In some embodiments, a photodiode 36 is configured to measure a power of a laser beam from the light source 4 transmitted through the source-side catheter 10. In some embodiments, a temperature sensor 38 is configured to measure an ambient temperature value of an ambient environment of the fiber optic catheter apparatus 32. The photodiode 36 is configured to measure the power of the laser beam based on the measured ambient temperature value.

As shown in FIG. 3, the catheter-side coupler 12 includes the catheter-side electronic processor 40 disposed therein and is programmed transmit information about the fiber optic catheter apparatus 32 to the light system 1 (e.g., the light source 4) in response to an electrical connection of the catheter-side coupler 12 with the source-side coupler 10 via the electrically conductive pins 34. At the same time, a physical configuration of the plurality of electrically conductive pins 34 encodes at least a portion of the information about the fiber optic catheter apparatus 32. The portion of the information that can be encoded by the physical configuration of the pins 34 is typically a subset of the information that can be transmitted electrically, because the pins 34 can only encode an N-bit binary value where N is the number of pins 34. As an example, for a six-pin connection, the 6-bit binary value can assume any one of 2^{N}=64 possible values. By contrast, the information that can be conveyed electrically is limited only by a data storage capacity of the catheter-side electronic processor 40 which can be on the order of kilobits, megabits, or more. Hence, the electrical transmission of the (complete) information is preferable over the mechanical transmission of (typically a subset of) the information; however, if the light source 4 to which the catheter 2 is connected is a model that is not designed to receive the information electrically then a sufficient subset of the information can be conveyed by the mechanical pin configuration (e.g. this can convey the catheter type, from which the light source 4 can extract operating parameters using a look-up table). For example, the catheter-side electronic processor 40 is configured to detect a configuration of the electrically conductive pins 34 that engage the mechanical switches 16 via signals generated by the electrical lines 22 of the engaged mechanical switches 16 to determine an identification of a catheter type of the catheter 2. To do so, if the catheter-side coupler 12 includes, for example, six pins 34 (i.e., “pin 1” through “pin 6”), then a physical configuration of having pins 1, 2, and 5 with corresponding mechanical switches 16 can identify the catheter 2 as having a first type. Similarly, a physical configuration of having pins 1, 3, and 6 with corresponding mechanical switches 16 can identify the catheter 2 as having a second, different type. Moreover, the number of pins 34 engaged with the switches 16 (i.e., 2 engaged switches 16, 4 engaged switches 4, and so forth) can identify the type of the catheter 2.

In some embodiments, the catheter-side electronic processor 40 is programmed to transmit the information about the fiber optic catheter apparatus 32 to the light system 1 via the electrically conductive pins 34. To do so, a serial communication circuit 42, such as an I2C communication circuit, is configured to communicate with the serial communication circuit 30 of the source-side coupler 10. In a further example, the source-side coupler 10 includes a wireless radio receiver or transceiver 44, and the catheter-side electronic processor 40 is programmed to transmit the information about the fiber optic catheter apparatus 32 to the light system 1 via the wireless radio receiver or transceiver 44.

In some embodiments, the catheter-side coupler 12 includes a memory 46 that stores memory content, including at least a catheter type of the fiber optic catheter apparatus 32. The transmitted information about the fiber optic catheter apparatus 32 includes the memory content. In some examples, the identification of the catheter type of the catheter 2 is transmitted to the memory 46 using a wired connection based on a number of mechanical switches 16 engaged by the electrically conducting pins 34. In some examples, upon determination of the identification of the type of the catheter 2, operating parameters of the catheter 2 are stored in the memory 46. These operating parameters can include, for example, an identification of the catheter 2, maximum fluence, minimum fluence, maximum rate, minimum rate, usage information, run time information and error information, among others. The memory 46 is illustrated as a separate component from the electronic processor 40; however, the memory alternatively may be integrated with the electronic processor 40, e.g. as a microprocessor with on-board memory.

As previously mentioned, the fiber optic catheter 2 is typically a single-use medical accessory due to sanitary considerations, e.g. the need to avoid cross-contamination between patients. In some embodiments, the catheter-side electronic processor 40 is programmed to set a flag indicating the catheter 2 has been used, which can be stored in the memory 46. Advantageously, this can prevent a catheter 2 from being used multiple times. The transmitted information about the fiber optic catheter apparatus 32 includes the set flag. If the set flag is received by the source-side coupler 10, then the source-side electronic processor 20 is programmed to prevent operation of the light source 4 from transmitting a laser beam. In another example, the catheter-side electronic processor 40 is configured to generate a fault condition flag indicative of when the catheter 2 has a fault condition, which can be stored in the memory 46. The fault condition flag may be a multi-bit flag that encodes information about the fault condition. The faulty catheter 2 can then be shipped back to the manufacturer which can read out the content of the memory 46 including the fault condition flag for use in determining why the catheter 2 failed. In another example, the catheter-side electronic processor 40 is configured to generate a validation code indicative of whether the source-side catheter 10 has been validated for use, which can be stored in the memory 46. For example, authorized catheter types can be assigned validation codes, and the light system 1 will then only drive the catheter if it supplies a recognized validation code. This can beneficially prevent the use of unauthorized counterfeit catheters that may be substandard and present a health risk to the patient.

The catheter-side coupler 12 also includes a power supply 48 for power to be supplied thereto. In some examples, the power supply 48 can include a DC-to-DC converter which reduces the power supplied to the source-side coupler 10 from 12 Volts to 3.3 Volts. This is done in order to ensure that the source-side coupler 10 has plenty of power, even if the conductivity of the pins 34 degrades over time. In another embodiment, the electrical wires 22 can be configured to supply power to the fiber optic catheter apparatus 32.

In other embodiments, the catheter-side electronic processor 40 is programmed to perform signal condition operations on a signal generated by the photodiode 36, including peak detection, and on the temperature value measured by the temperature sensor 38. The catheter-side electronic processor 40 can also include an analog-to-digital A/D) converter (not shown) in order to further process such signals.

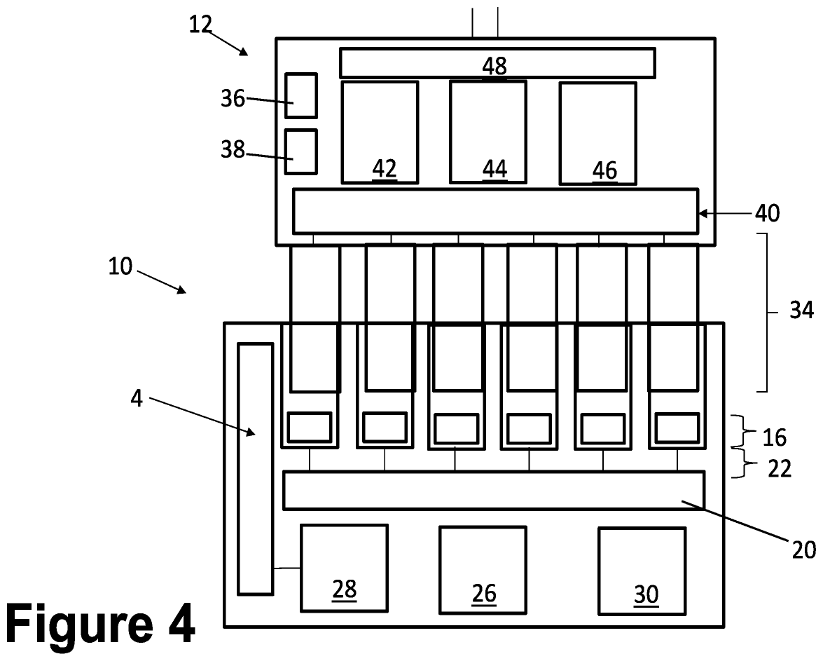

FIG. 4 shows a connection between the source-side coupler 10 and the catheter-side coupler 12. FIG. 4 shows that the pins 34 of the catheter-side coupler 12 engage switches 16 of the source-side coupler 10.

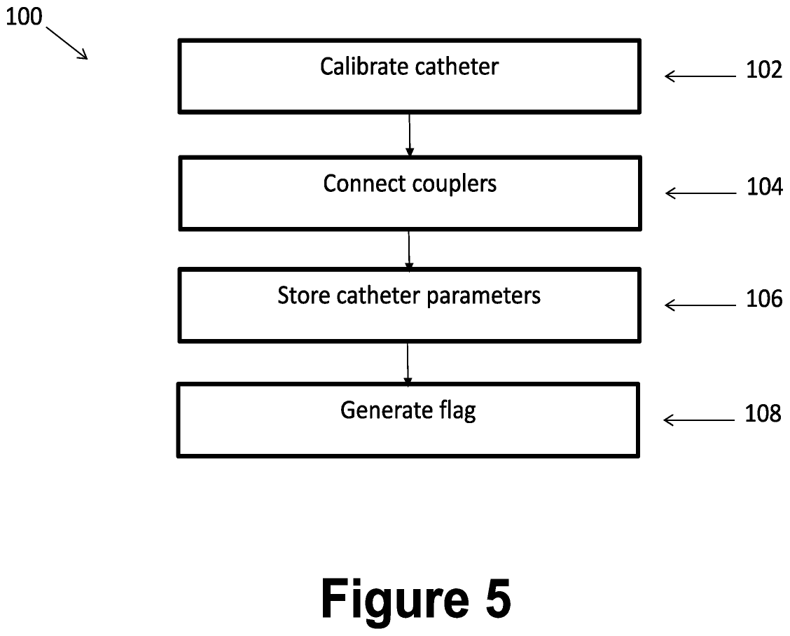

FIG. 5 shows an example of a flowchart showing a catheter method 100 using the light system 1 and the fiber optic catheter apparatus 32. At an operation 102, one or more catheters 2 can be calibrated, and a calibration factor can be generated for each type of catheter and stored in the memory 46 of the catheter-side coupler 12. Additionally, a unique catheter ID, min/max fluence, min/max rate, and run time parameters can be generated and stored. At an operation 104, the catheter-side coupler 12 is connected to the source-side coupler 10 and a communication pathway is established via the serial communication circuits 30, 42. At an operation 106, the parameters stored in the memory 46 are transmitted to the source-side coupler 10. In an optional operation 108, one or more flags can be generated (e.g., a previous use flag, a technical issue flag, and so forth) and stored in the memory 36 during use of the catheter 2.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A source-side coupler for injecting light from an associated light source into an associated fiber optic catheter via a connection of the source-side coupler with a catheter-side coupler of the associated fiber optic catheter, the source-side coupler comprising:
an optical output aperture configured to inject light from the associated light source into the associated fiber optic catheter via the connection;
a plurality of mechanical switches configured to be engaged by pins of the catheter-side coupler during the connection;
at least two electrical lines; and
an electronic processor programmed to receive information from the associated fiber optic catheter via the connection by:
electrically receiving the information if the connection establishes an electrical connection between the electronic processor and the associated fiber optic catheter via the at least two electrical lines, or
mechanically receiving the information based on which mechanical switches of the plurality of mechanical switches are engaged by pins of the catheter-side coupler if the connection does not establish an electrical connection between the electronic processor and the associated fiber optic catheter via the at least two electrical lines.

2. The source-side coupler of claim 1, wherein the electronic processor is programmed to electrically receive the information via the at least two electrical lines.

3. The source-side coupler of claim 1, further comprising a wireless radio receiver or transceiver, wherein the electronic processor is programmed to electrically receive the information via the wireless radio receiver or transceiver.

4. A light system for injecting light into an associated fiber optic catheter, the light system comprising:
a light source; and
a source-side coupler as set forth in claim 1.

5. A fiber optic catheter apparatus, comprising:
a catheter including an optical fiber or optical fiber bundle terminating at an optical output aperture;
a catheter-side coupler including a plurality of electrically conductive pins; and
an electronic processor programmed transmit information about the fiber optic catheter apparatus to an associated light source in response to an electrical connection of the catheter-side coupler with a source-side coupler of the associated light source via the electrically conductive pins;
wherein a physical configuration of the plurality of electrically conductive pins encodes at least a portion of the information about the fiber optic catheter apparatus.

6. The fiber optic catheter apparatus of claim 5, wherein the electronic processor is programmed to transmit the information about the fiber optic catheter apparatus to the associated light source via the electrically conductive pins.

7. The fiber optic catheter apparatus of claim 6, wherein the information is transmitted via the electrically conductive pins using a serial communication circuit.

8. The fiber optic catheter apparatus of claim 6, further comprising:
a wireless radio transmitter or transceiver, wherein the electronic processor is programmed to transmit the information about the fiber optic catheter apparatus to the associated light source via the wireless radio receiver or transceiver.

9. The fiber optic catheter apparatus of claim 5, wherein the electrically conductive pins are configured to engage mechanical switches of the source-side coupler, the mechanical switches including electrical wires; and
the electronic processor is configured to detect a configuration of the electrically conductive pins that engage the mechanical switches via signals generated by the electrical wires of engaged mechanical switches to determine an identification of a catheter type of the fiber optic catheter apparatus.

10. The fiber optic catheter apparatus of claim 5, further including:
a memory storing memory content including at least a catheter type of the fiber optic catheter apparatus;
wherein the transmitted information about the fiber optic catheter apparatus includes the memory content.

11. The fiber optic catheter apparatus of claim 10, wherein the identification of the catheter type of the fiber optic catheter apparatus is transmitted to the memory using a wired connection based on a number of mechanical switches engaged by the electrically conducting pins.

12. The fiber optic catheter apparatus of claim 10, wherein, upon determination of the identification of the type of the fiber optic catheter apparatus, operating parameters of the catheter are stored in the memory.

13. The fiber optic catheter apparatus of claim 10, wherein the electronic processor is configured to set a flag indicating the catheter has been used, wherein the flag is stored in the memory.

14. The fiber optic catheter apparatus 32 of claim 13, wherein the transmitted information about the fiber optic catheter apparatus includes the set flag.

15. The fiber optic catheter apparatus of claim 10, wherein the electronic processor is configured to generate a fault condition flag indicative of when the catheter has a fault condition, wherein the fault condition flag is stored in the memory.

16. The fiber optic catheter apparatus 3 of claim 10, wherein the electronic processor is configured to generate a validation code indicative of whether the source-side coupler has been validated for use, wherein the validation code is stored in the memory.

17. The fiber optic catheter apparatus of claim 5, further including:
a photodiode configured to measure a power of a laser beam transmitted through the source-side coupler.

18. The fiber optic catheter apparatus of claim 17, further including:
a temperature sensor configured to measure an ambient temperature value of an ambient environment of the fiber optic catheter apparatus;
wherein the photodiode is configured to measure the power of the laser beam based on the measured ambient temperature value.

19. The fiber optic catheter apparatus of claim 5, wherein the fiber optic catheter apparatus is configured to receive power from electrical lines of the source-side coupler of the associated light source.

20. A light system for injecting light into a fiber optic catheter, the light system comprising:
a light source;
a fiber optic catheter apparatus, comprising:
a catheter including an optical fiber or optical fiber bundle terminating at an optical output aperture;
a catheter-side coupler including a plurality of electrically conductive pins; and
a fiber optic catheter electronic processor disposed in the catheter-side coupler and programmed transmit information about the fiber optic catheter apparatus to the light source;
wherein a physical configuration of the plurality of electrically conductive pins encodes at least a portion of the information about the fiber optic catheter apparatus; and
a source-side coupler for injecting light from the light source into the fiber optic catheter apparatus via a connection of the source-side coupler with the catheter-side coupler, the source-side coupler comprising:
an optical coupler configured to inject light from the light source into the fiber optic catheter via the connection;
a plurality of mechanical switches configured to be engaged by the electrically-conductive pins of the catheter-side coupler during the connection;
at least two electrical lines; and
a source-side electronic processor programmed to receive information from the fiber optic catheter via the connection by:
electrically receiving the information if the connection establishes an electrical connection between the source-side electronic processor and the fiber optic catheter via the at least two electrical lines, or
mechanically receiving the information based on which mechanical switches of the plurality of mechanical switches are engaged by pins of the catheter-side coupler if the connection does not establish an electrical connection between the electronic processor and the fiber optic catheter via the at least two electrical lines.
